(19) United States
(12) Patent Application Publication
(10) Pub. No.: US 2023/0092566 A1
(43) Pub. Date: Mar. 23, 2023

(54) SENSING PHYSIOLOGICAL ACTIVITY

(71) Applicant: Jonathan Kamler, Jamaica, NY (US)

(72) Inventor: Jonathan Kamler, Jamaica, NY (US)

(21) Appl. No.: 17/946,737

(22) Filed: Sep. 16, 2022

Related U.S. Application Data
(63) Continuation of application No. 17/592,300, filed on Feb. 3, 2022.
(60) Provisional application No. 63/245,594, filed on Sep. 17, 2021.

(51) Int. Cl. A61B 5/021; G01N 29/34; A61B 5/00

(52) U.S. Cl. CPC A61B 5/02125; G01N 29/34; A61B 5/6833

(57) ABSTRACT

The disclosure describes devices for monitoring physiological activity (e.g., a pulse). These devices can include: a detector configured to detect sound; an ultrasound system to detect fluid flow; an adhesive to attach a detector to skin of a patient; an adhesive to attach a probe to skin of a patient; and a speaker to amplify or communicate detected sound or flow.

### TECHNICAL FIELD

The present disclosure relates generally to systems and methods for sensing and relaying physiological activity.

### BACKGROUND

Pulse measurement devices and cardiac monitors are used in the medical industry to assess heart rate and rhythm data of a user. Stethoscopes are used to assess cardiac, pulmonary, gastrointestinal, and other information about the human body. They can be used in the management of patient health. Some devices monitor and store health data to assist in the monitoring of patient conditions and managing the progression of an illness. Such devices can also be used in the sport and exercise industries. Coaches and health professionals may want to monitor and analyze an athlete's physical activities.

### SUMMARY

This specification describes methods and systems for sensing a pulse, sound, or other indicator of physiological activity. These methods and systems enable efficiently sensing and relaying a pulse, sound, or other physiological signal to a user while limiting the electronics or other hardware necessary to do so.

In an aspect, a device for monitoring a pulse includes a detector, an adhesive to attach the detector to skin of a patient, and a speaker to communicate information detected by the detector. As used herein, “detector” may have different meanings for different embodiments. In some cases, its function may be based on interaction with sound (e.g. audible sound, ultrasound) or vibrations. For example, the detector may function as a probe that emits/receives ultrasound waves. As another example, the detector may function as a resonator. Embodiments can include one or more of the following features.

In some embodiments, the detector can send or receive ultrasound waves.

In some embodiments, the detector uses Doppler technology.

In some embodiments, the detector is a resonator.

In some embodiments, the speaker is configured to continuously amplify the sound detected by the detector.

In some embodiments, the speaker is configured to produce an alarm when the detector does not detect sound above a certain threshold volume or with certain characteristics.

In some embodiments, the detector is a plastic disc.

In some embodiments, the detector is a hollow cup.

In some embodiments, the device includes indicia for correct placement of the detector on the skin of the patient.

In some embodiments, the device includes a sound conducting gel.

In some embodiments, the device mechanically relays sound from the detector to the speaker.

In some embodiments, the device electronically relays sound from the detector to the speaker.

In some embodiments, sound is relayed without the need for a speaker.

In some embodiments, the detector includes a microphone.

In some embodiments, the device includes transmitters and receivers operable to send and detect reflected ultrasonic waves, a transducer operable to convert electrical signals into ultrasonic waves and convert reflected waves into electrical signals, and a processor operable to control the transmitters, receivers, and transducers to send an ultrasonic wave, detect a reflection of the ultrasonic wave, convert the reflection of the ultrasonic wave into an electrical signal, and relay the electrical signal to a communication apparatus such as a speaker or display screen to provide information related to the ultrasonic waves (e.g, to communicate pulse status or respiratory status).

In an aspect, a method of monitoring a pulse includes attaching a detector to skin of a patient and using a speaker to communicate information detected by the detector. Embodiments can include one or more of the following features.

In some embodiments, attaching the detector to the skin of the patient includes using an adhesive to attach the detector to the skin of the patient.

In some embodiments, the method includes sending or receiving ultrasound waves.

In some embodiments, the method includes the use of Doppler technology.

In some embodiments, the method includes use of a resonator.

In some embodiments, the method includes mechanically relaying the sound detected by the detector to the speaker.

In some embodiments, the method includes electronically relaying the sound detected by the detector to the speaker.

In some embodiments, the method includes using a microphone to relay the sound detected by the detector to the speaker.

In some embodiments, the speaker continuously amplifies the sound detected by the detector.

In some embodiments, sound is relayed without the need for a speaker.

In some embodiments, the method includes producing an alarm with the speaker when sound meeting particular criteria (e.g., being below a threshold volume) is detected by the detector.

In some embodiments, attaching the detector to the skin of the patient includes attaching the detector to an extremity of the patient.

In some embodiments, the method includes sending an ultrasonic wave, detecting a reflection of the ultrasonic wave, converting the reflection of the ultrasonic wave into an electrical signal, and relaying the electrical signal to the speaker to signify a pulse.

These systems and methods can provide timely awareness of a change in pulse status, for example, from being palpable to being non-palpable, and vice-versa. Algorithms for the care of critically ill patients often change depending on whether a pulse is palpable or not. When a change in pulse status occurs, rapid awareness of this change (e.g., via an “alarm”) by medical providers can potentially alter medical management and lead to more optimized patient outcomes. Other advantages of various embodiments may include: awareness of pulse status with limited equipment; portable, easily applied design; determination of pulse status with limited need for provider judgement; awareness of pulse status with reduced need for active involvement of providers, facilitating focus on other aspects of care or on other patients requiring more immediate attention. An advantage of the proposed devices and methods is that they can, in some embodiments, provide real-time information about pulse status. For example, in cardiac arrest patients, a provider might regularly assess whether a pulse is palpable. This may inform whether chest compressions should be continued. However, without real-time continuous detection, there may be a time delay between when a pulse becomes nonpalpable or becomes palpable, and when the provider is aware of the change and able to act accordingly. This may delay application of an optimal intervention. As another example, patients may present in atrial fibrillation and be treated to restore a regular rhythm. Continuous awareness of the regularity or irregularity of the pulse can indicate in real time whether a treatment is effective and whether additional treatment is needed. In both medical and non-medical (e.g. home) settings, it may be useful for a provider or patient to know exactly when a change in pulse regularity occurs. For example, a patient whose rhythm becomes irregular (e.g., reflecting atrial fibrillation) may benefit from knowing when the change occurred, so that subsequent medical management can be adjusted according to the duration over which the patient's rhythm was irregular.

In some embodiments, the systems and methods can provide information about the respiratory status of a patient. For example, if placed in proximity to the patient's lung, the device can continuously amplify breath sounds and allow providers to become rapidly aware of changes in breath sounds. As a specific example, a patient may present with an asthma exacerbation, be found to have “wheezing” breath sounds initially, and be provided with nebulized albuterol to alleviate the patient's respiratory symptoms. Using an embodiment, a provider can be made aware of real-time changes to the patient's breath sounds. Perhaps the wheezing will diminish or disappear after the albuterol treatment, and perhaps the wheezing will return several minutes later as the effect wears off. Rapid awareness of such changes in breath sounds may improve the delivery of care to the patient. Other examples/embodiments include: monitoring the breath sounds of a patient on a ventilator to help determine whether a pneumothorax develops; monitoring the breath sounds of a patient with a pneumothorax to assess whether lung re-expansion occurs after treatment; monitoring stridor in patients with unstable airway obstructions; monitoring a patient's ventilatory adequacy (e.g., respiratory rate); monitoring the bowel sounds of patients after surgery, to determine the extent of ileus and the effectiveness of bowel motility treatments, or whether obstruction develops; monitoring for turbulent blood flow or a “bruit” at the carotid arteries to assess for stenosis; monitoring dorsalis pedis pulses in the feet to assess for progression of vascular disease or recanalization after surgery. In some cases, more than one device may be attached to the patient simultaneously, for example to monitor breath sounds in multiple locations or blood flow in multiple extremities. In some cases, the device(s) may be worn over long periods of time or indefinitely.

As used herein, the term “pulse” and similar terminology may be used to refer to the pulsatile passage of blood flow in a vessel (e.g., artery or vein). In some cases, the “presence” of a pulse more specifically refers to situations in which such flow is palpable (e.g., can be felt by external application of fingers to the patient's skin in the vicinity of an artery). In some cases, a “pulse” can be identified by other means (e.g., visualization of cardiac activity or Doppler measurement of periodic fluid flow). In some embodiments, these systems and methods can be used to detect cardiac activity directly; for example, by detecting movement at the heart rather than at blood vessels. Though this may not strictly constitute the detection of a “pulse”, it will be apparent to those skilled in the art that this approach can be used in such cases and the use of the term “pulse” herein should not be construed as a limitation.

Other implementations are within the scope of the claims.

### DESCRIPTION OF DRAWINGS

FIG. 1 is a diagram of a pulse sensor.

FIG. 2 is an illustration of a pulse sensor on a neck.

FIG. 3 is an illustration of a pulse sensor on a leg.

FIG. 4 is an illustration of a pulse sensor on a finger.

FIG. 5 is an illustration of a pulse sensor in the vicinity of a vessel.

### DETAILED DESCRIPTION

This specification describes methods and systems for sensing cardiac and other physiological activity, for example, a pulse. These methods and systems enable efficiently sensing and relaying cardiac activity, for example, information about a pulse to a user while minimizing the electronics necessary to do so. The devices or methods can relay information about the cardiovascular status of a patient to someone providing medical care to the patient. Awareness of cardiovascular status over time can provide critical information affecting medical care and the health of the patient. Detecting a “pulse” and when it changes is performed routinely, for example in the diagnosis of cardiac arrest and to assess perfusion in a limb. Timely awareness of a pulse change (e.g., changing between palpable and non-palpable; changing among periodic blood flow, non-periodic blood flow, and/or no flow; changing between regular and irregular) can be vital in medical decision-making. Detecting a pulse is often used as a limited proxy for cardiac activity, and other approaches to measuring cardiovascular activity (e.g., palpation or visualization or the hearing of a beating heart) can also provide, in some cases, equivalent information. For example, “feeling” a pulse (e.g., using a finger against the patient's skin), listening for a heartbeat or turbulent flow (e.g., with a stethoscope), visualizing cardiac motion (e.g., with ultrasound), and visualizing blood flow (e.g., with Doppler) represent approaches to assessing cardiovascular function that can be replicated in different embodiments. The devices and methods can be applied to such cases where it is not strictly a “pulse” that is being detected. Different embodiments may have different thresholds for pulse “detection”, which may correspond to different cardiovascular states. A pulse that is “palpable” (e.g., using a provider's fingers applied near an artery of the patient) or “pulsatile” (e.g., as detected in a limb artery using Doppler ultrasound) is often considered evidence of organized cardiac activity capable of producing blood flow around the body. Such terminology is used herein to generally suggest a threshold change in cardiovascular status. For example, in various embodiments, pulse detection may involve producing audible sound correlating with Doppler ultrasound measurement of blood flow; visual display of blood flow in a vessel based on ultrasound imaging; relaying the sound of a heartbeat as detected by a resonator; communicating information about mechanical or electrical indicators of blood flow; and other formats. The devices and methods may be useful in acute or critical care settings, such as during CPR, as well as in other settings, such as home monitoring or for long-term monitoring (e.g. over days, months, years).

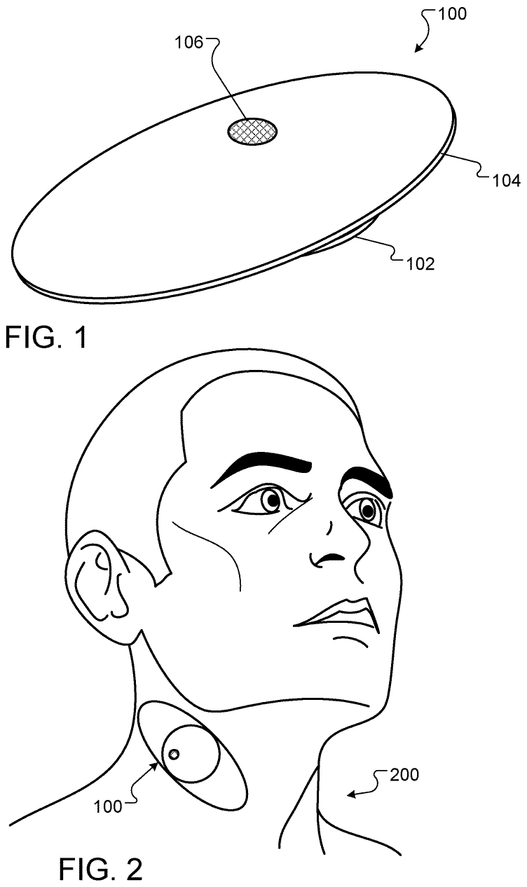

FIG. 1 illustrates a pulse sensor 100 that includes a detector 102. The illustrated detector is a disk that can be placed on the body of a patient. In some embodiments, the detector incorporates ultrasound or Doppler technology. In some embodiments, the detector is a resonator. When a resonator is placed on the body, it vibrates or deforms with the pulse or a sound (e.g., heartbeat, breath sounds) of a patient, like a stethoscope. The detector 102 can be any of a variety of tools or materials that facilitates the detection or measurement of a pulse or sound, for example an ultrasound probe, a Doppler probe, a stethoscope, a stethoscope diaphragm, a stethoscope bell, or a microphone. The detector 102 can be a flat disk, or the detector can have a curved shape to form a hollow cup. The detector 102 can be, e.g., plastic or metal. In some implementations, the detector 102 includes a sound conducting gel so that the detector 102 can better sense the pulse of or a sound from the patient.

Devices for sensing a pulse can include ultrasound or Doppler probes, electronic microphones, piezoelectric materials, or pressure sensors. Such devices, with appropriate electronics, can detect features representative of a pulse, such as pulsatile flow or soft tissue deformation, which can in turn be used to produce sounds or images that can be understood by medical providers (note that “medical providers” and similar terminology may refer to anyone providing care to a patient). For example, an ultrasound probe can be oriented to direct ultrasound waves toward an artery, and can produce a sound signal corresponding to the periodic passage of blood flow in the artery or movement of the vessel walls. Changes in the blood flow, such as a change in the periodicity of flow (including cessation or resumption of flow), can be communicated to medical providers and used to inform medical management.

The pulse sensor 100 also includes adhesive 104 to hold the detector to the skin of the patient. The pulse sensor 100 includes a speaker 106 that can relay sound detected by the detector 102 or provide other information. For example, when the pulse sensor 100 is placed on the skin of a patient, the detector (e.g., a resonator) 102 can vibrate with the heartbeat or pulse of the patient, and the speaker 106 can amplify the vibration of the detector to yield an audible signal or play a sound synchronized with the vibration of the detector. It can be advantageous for the pulse sensor 100 to continuously relay the heartbeat or pulse of the patient because the pulse could change, stop, or start. Because the pulse sensor 100 can continuously relay the pulse status of the patient, this approach can reduce the need for a user or a medical professional to check for the pulse periodically or worry about whether a loss of pulse may be occurring.

The speaker 106 is activated by a signal based on detection of the vibration of the detector 102. In some systems, the speaker 106 amplifies the sounds detected by a microphone and/or produces artificial sounds synchronized with heartbeats detected. For example, the device can relay or amplify the sound directly from a resonator, from a microphone, or from electronic signals (e.g. Doppler ultrasound signals).

In some systems, the sound is mechanically relayed from the detector to the speaker. In some systems, the detector 102 is wired to the speaker 106 and includes a transducer so that the speaker 106 can electronically detect the vibration of the detector (e.g., a resonator) 102. In these systems, the sound is electronically relayed from the detector to the speaker. Some systems do not include a speaker 106 because the detector 102 detects and amplifies sound (e.g., similar to a stethoscope). In some implementations, the speaker 106 creates a different sound, e.g., an alarm, when certain parameters are met or fail to be met; for example, when the detector 102 does not detect a pulse. The speaker 106 can also create a different sound when there is a significant drop in pulse. In some implementations, the speaker 106 creates a sound when there is a return of pulse after previously detecting no pulse. This can be useful, for example, in indicating successful CPR or successful vessel recanalization or limb replantation. The speaker 106 can create a sound when there is any significant change (i.e., above a certain threshold) in pulse rate, rhythm, strength, etc.

Some pulse sensors use the Doppler effect, which includes converting electrical signals into ultrasonic waves, and converting the reflected waves into electrical signals to detect a pulse. Using the Doppler effect to detect a pulse may require more electronics and processing power. In this approach, the pulse sensor includes transmitters and receivers to send and detect reflected ultrasonic waves. The pulse sensor can also include a transducer to convert electrical signals into ultrasonic waves and convert reflected waves into electrical signals signifying a pulse. The speaker can then play sounds synchronized with the pulse. A processor or computer can control the transmitters, receivers, transducers, and speaker to relay the pulse. For example, the processor can control the transmitters to send an ultrasonic wave, control the receivers to detect a reflection of the ultrasonic wave, control the transducers to convert the reflection of the ultrasonic wave into an electrical signal, and control the speaker to relay the electrical signal to signify a pulse. The pulse sensor 100 can also send a signal to an external computer or processor (e.g., wirelessly).

In some implementations, the processor of the pulse sensor 100 analyzes the reflected waves to determine whether the reflected waves are indicative of a pulse. In other implementations, the processor of the pulse sensor 100 does not analyze, or minimally analyzes, the reflected waves, and primarily relays and amplifies information from the reflected waves through the speaker. Reducing the processing needs can reduce the electronics and memory which would otherwise be required for analyzing the reflected waves. Relaying detected sounds with minimal analysis or transformation also allows a medical provider to hear intricacies in the detected sounds. For example, detected sounds are relayed that are not necessarily indicative of a pulse, but are otherwise helpful for a medical provider.

The pulse sensor 100 can sense such features of the patient's cardiovascular system by means including, for example, ultrasound, Doppler, mechanical, or electrical signals. For example, Doppler probes are routinely used to detect pulses in limbs or heartbeats in fetuses. The pulse sensor 100 can detect pulses or cardiac activity (e.g., carotid pulse when placed on the neck; heartbeat when placed over the chest) with ultrasound/Doppler technology incorporated into the pulse sensor 100. Both sound and visual information can be used. For example, a Doppler probe, with appropriate electronics (e.g., transmitters, transducers), can be used to produce a sound corresponding to the blood flow in a blood vessel. As another example, an ultrasound probe can produce an image of blood vessels (or of the heart) and visually demonstrate pulsatile flow (or beating) therein. In some embodiments, ultrasound probes without Doppler technology can be used.

Some systems measure mechanical deformation or pressure over the skin, for example to assess the presence of a palpable pulse. Sound produced by the body (e.g., a heartbeat, turbulent blood flow) can be detected, measured, relayed, and/or amplified by a resonator. Additionally, electromagnetic field changes can be measured to assess local blood flow. In some embodiments, the pulse sensor 100 is equipped (e.g., with Hall sensors) to detect changes in electromagnetic fields. In some examples, systems measure detectable changes in color, light scattering, impedance, or conductivity. These in turn can be indicative of physiological information, such as blood flow at a given time. The pulse sensor 100 can be equipped with optical sensors, Hall sensors, photoplethysmography systems, and/or transducers to detect physiological states or changes. Many of these approaches would be considered noninvasive. In some embodiments, the pulse sensor 100 includes a pressure sensor that detects movement of soft tissue corresponding to the presence of a pulse. This can include detection of a “palpable” pulse.

In some implementations, the pulse sensors 100 relay “less processed” information, such as sound corresponding to periodic blood flow, or imagery of blood vessels. Alternatively or additionally, the pulse sensors 100 can relay “more processed” information, such as an indication that there is adequate blood flow or not. However, this would require additional computation (e.g., comparing the detected blood flow to a threshold value). The pulse sensor 100 can additionally provide alerts to important changes (e.g., pulsatile/palpable flow no longer present, or now present). The pulse sensor 100 may continuously relay information, which a provider can monitor while caring for the patient.

Some pulse sensors 100 perform useful calculations on the basis of acquired data, for example to assess how “strong” the pulse is or to guide provider interventions (e.g., the quality of chest compressions). The pulse sensor 100 can be an adhesive sticker as described above, or in other implementations the pulse sensor 100 can take other forms, for example, as a cuff applied around a limb.

Some approaches include applying a pulse sensor to an area of the patient's body where one would expect a pulse to be palpable in normal health. For example, upon encountering an unresponsive patient, a provider can apply the device to the neck near the carotid artery with the pulse sensor indicating whether a periodic or a palpable pulse is present. Because the pulse sensor can provide continuous communication (e.g., continuous sound), it will be immediately apparent to a provider if, for example, the pulse is initially present and then becomes undetectable, or vice versa.

FIG. 2 illustrates this exemplary use of the pulse sensor of FIG. 1. The pulse sensor 100 has been placed on the neck 200 of a patient, e.g., near the carotid artery, so that the pulse sensor 100 can continuously detect a pulse in the neck 200 of the patient. For example, the pulse sensor 100 can be applied to the neck of an unresponsive patient by a provider. Some pulse sensors 100 use Doppler ultrasound technology to detect pulsatile blood flow in the neck (e.g., in the carotid artery). The pulse sensor 100 has a speaker that produces sound corresponding to the blood flow. The provider listens for the presence or absence (or a change) of this sound to determine, for example, whether there is blood flow to guide acute management. Additionally, the pulse sensor 100 creates an alarm when a change in blood flow occurs, e.g., the pulse becomes detectable or ceases to be detectable.

For example, the pulse sensor 100 can be used during CPR to determine whether a pulse is present, or detect whether chest compressions are sufficient to produce blood flow. While performing CPR with the pulse sensor 100 placed on the neck 200 of the patient, the speaker of the pulse sensor 100 can make sound synchronized with detected blood flow in the neck 200. If no flow is detected, the speaker 106 makes a different sound, e.g., an alarm, to notify the person providing CPR that there is no pulse. The pulse sensor can be used to determine whether there is blood flow or a pulse when chest compressions are withheld, which may be indicative of the resumption of a heartbeat.

Some pulse sensors are configured to provide instructions for use to an untrained person. For example, a person performing CPR may be an amateur instead of a trained medical professional. These pulse sensors can include, for example, indicia to provide instructions for correctly placing the sensor over the carotid artery. Alternatively or additionally, the speaker of the pulse sensor can provide instructions to the person performing CPR. For example, the speaker may create different sounds to notify the person performing CPR that the chest compressions are too slow or too fast. In another example, the speaker may provide a target compression rate for the person performing CPR to replicate. Increasing the effectiveness of the person performing CPR through provided instructions can increase the likelihood of survival of the patient receiving CPR.

In another example, the pulse sensor 100 can be applied to a critically ill patient so that providers are immediately aware if the patient's pulse is lost. The speaker 106 can alarm to notify medical providers that there is no pulse.

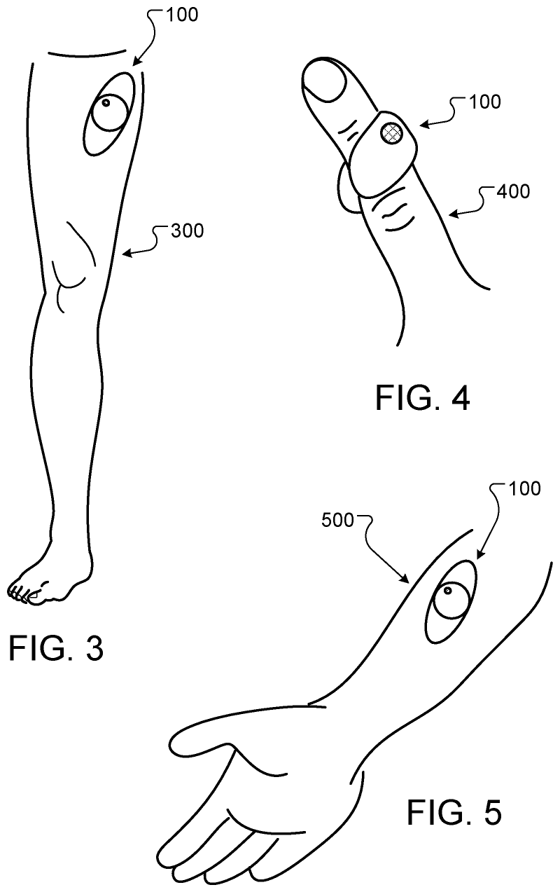

FIG. 3 illustrates another exemplary use of the pulse sensor of FIG. 1. In this application, the pulse sensor 100 is placed on the leg 300 of a patient to sense a pulse in the leg 300 of the patient. The leg 300 has major arteries, e.g., the femoral artery, that are useful for detecting a pulse. Other major arteries in the body can also be used for detecting a pulse. In some cases, it is beneficial for there to be no pulse. For example, a tourniquet may need to be placed around the leg 300 to prevent blood flow to the leg after a traumatic injury. The sensor 100 can be placed distally to the tourniquet to confirm that there is no pulse (i.e., no blood flow) in the leg 300 below the tourniquet. In some embodiments, the pulse sensor can be used to distinguish blood vessels with and without pulsatile flow, e.g. arteries and veins, respectively.

FIG. 4 illustrates another exemplary use of the pulse sensor of FIG. 1. In this application, the pulse sensor 100 is placed on a finger 400 of a patient to sense a pulse in the finger 400. It is often more difficult to sense a pulse in certain smaller parts of the extremities. However, there are times that it is important to detect a pulse in an extremity (e.g., the finger 400). In difficult situations, the Doppler effect can be utilized to detect pulses in smaller arteries. For example, a patient may have a finger 400 reattached to the hand. Once reattached, the finger 400 should have blood flow if reattached correctly. A pulse sensor 100 utilizing the Doppler effect can confirm whether vasculature in the finger was correctly reattached to vasculature in the hand. As another example, after major traumatic injury, it may be helpful to determine whether a damaged limb has intact pulses distal to the site of injury. This may assist with assessment of vascular injury, the likelihood of rescuing the limb, and the urgency of potential medical (e.g. surgical) intervention or of rescue efforts. Similar techniques can also be used in other contexts in which “local” pulse or blood flow detection is important, such as organ transplantation. For example, after surgical placement of an organ in a recipient, it may be valuable to assess blood flow to the organ using a pulse sensor.

FIG. 5 illustrates another exemplary use of the pulse sensor of FIG. 1. In some examples, it can be beneficial to detect a pulse in a surgically manipulated or created vessel within the body. For example, in FIG. 5, the pulse sensor 100 is placed on an arm 500. Inside the arm 500, there may be a surgically created vessel (e.g., a dialysis fistula) attached to the cardiovascular system. Dialysis fistulas are typically within an arm of the patient and are useful in providing strong blood flow for dialysis. Problems with the fistula, including cessation of flow or the development of a blood clot, may render it difficult or impossible to perform dialysis, and may require rapid surgical repair or other major interventions. Thus, verification of flow through the fistula, or determination of the presence of a local pulse or thrill, is frequently performed and useful for the optimal care of the patient.

Other uses are imaginable for the pulse sensor 100. For example, the pulse sensor 100 can be applied to the abdomen of a pregnant patient to continuously detect the heartbeat of a fetus. In another example, the pulse sensor 100 can be used in conjunction with an occlusive cuff (e.g. “blood pressure cuff”) that can change the blood flow in the blood vessels of a limb, for example by stopping flow or causing the flow to become turbulent.

In some embodiments, the systems and methods can provide information about the respiratory status of a patient. For example, if placed in proximity to the patient's lung, the device can continuously amplify breath sounds and allow providers to become rapidly aware of changes in breath sounds. As a specific example, a patient may present with an asthma exacerbation, be found to have “wheezing” breath sounds initially, and be provided with nebulized albuterol to alleviate the patient's respiratory symptoms. Using an embodiment, a provider can be made aware of real-time changes to the patient's breath sounds. Perhaps the wheezing will diminish or disappear after the albuterol treatment, and perhaps the wheezing will return several minutes later as the effect wears off. Rapid awareness of such changes in breath sounds may improve the delivery of care to the patient. Other examples/embodiments include: monitoring the breath sounds of a patient on a ventilator to help determine whether a pneumothorax develops; monitoring the breath sounds of a patient with a pneumothorax to assess whether lung re-expansion occurs after treatment; monitoring stridor in patients with unstable airway obstructions; monitoring a patient's ventilatory adequacy (e.g., respiratory rate); monitoring the bowel sounds of patients after surgery, to determine the extent of ileus and the effectiveness of bowel motility treatments, or whether obstruction develops; monitoring for turbulent blood flow or a “bruit” at the carotid arteries to assess for stenosis; monitoring dorsalis pedis pulses in the feet to assess for progression of vascular disease or recanalization after surgery. In some cases, more than one device may be attached to the patient simultaneously, for example to monitor breath sounds in multiple locations or blood flow in multiple extremities. In some cases, the device(s) may be worn over long periods of time or indefinitely.

The computer is intended to include various forms of digital computers, such as printed circuit boards (PCB), processors, digital circuitry, or otherwise parts of a system for determining physiological activity. Additionally the system can include portable storage media, such as Universal Serial Bus (USB) flash drives. For example, the USB flash drives may store operating systems and other applications. The USB flash drives can include input/output components, such as a wireless transmitter or USB connector that may be inserted into a USB port of another computing device.

The computer can include a processor, a memory, a storage device, and an input/output device (for displays, input devices, example, sensors, valves, pumps). Each of the components are interconnected using a system bus. The processor is capable of processing instructions for execution within the computer. The processor may be designed using any of a number of architectures. For example, the processor may be a CISC (Complex Instruction Set Computers) processor, a RISC (Reduced Instruction Set Computer) processor, or a MISC (Minimal Instruction Set Computer) processor.

In one implementation, the processor is a single-threaded processor. In another implementation, the processor is a multi-threaded processor. The processor is capable of processing instructions stored in the memory or on the storage device to display graphical information for a user interface on the input/output device.

The memory stores information within the computer. In one implementation, the memory is a computer-readable medium. In one implementation, the memory is a volatile memory unit. In another implementation, the memory is a non-volatile memory unit.

The storage device is capable of providing mass storage for the computer. In one implementation, the storage device is a computer-readable medium. In various different implementations, the storage device may be a floppy disk device, a hard disk device, an optical disk device, or a tape device.

The input/output device provides input/output operations for the computer. In one implementation, the input/output device includes a keyboard and/or pointing device. In another implementation, the input/output device includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

This specification describes devices, methods, and systems for sensing a pulse, blood flow, heartbeat, breath sounds, other sounds and other mechanical or electric properties of physiological systems. It will be appreciated that various changes may be made by those skilled in the art without departing from the spirit and scope of this disclosure.

What is claimed is:

1. A device for monitoring a pulse, the device comprising:
a detector configured to detect sound;
an adhesive to attach the detector to skin of a patient; and
a speaker to amplify sound detected by the detector.

2. The device of claim 1, wherein the speaker is configured to continuously amplify the sound detected by the detector.

3. The device of claim 1, wherein the detector is a resonator.

4. The device of claim 1, wherein the speaker is configured to produce an alarm when the detector does not detect sound above a certain threshold volume or with certain characteristics.

5. The device of claim 1, wherein the detector is a plastic disc.

6. The device of claim 1, wherein the detector is a hollow cup.

7. The device of claim 1, further comprising indicia for correct placement of the detector on the skin of the patient.

8. The device of claim 1, further comprising a sound conducting gel.

9. The device of claim 1, wherein the device mechanically relays sound from the detector to the speaker.

10. The device of claim 1, wherein the device electronically relays sound from the detector to the speaker.

11. The device of claim 10, wherein the detector further comprises a microphone.

12. A device for monitoring a pulse, the device comprising:
transmitters and receivers configured to send and detect reflected ultrasonic waves;
a transducer configured to convert electrical signals into ultrasonic waves and convert reflected waves into electrical signals;
a speaker;
an adhesive to attach the device to skin of a patient; and
a processor configured to control the transmitters, receivers, and transducer to send an ultrasonic wave, detect a reflection of the ultrasonic wave, convert the reflection of the ultrasonic wave into an electrical signal, and relay the electrical signal to the speaker to signify a pulse.

13. A method of monitoring a pulse, the method comprising:
attaching a detector to skin of a patient;
using a speaker to amplify a sound detected by the detector.

14. The method of claim 13, wherein attaching the detector to the skin of the patient comprises using an adhesive to attach the detector to the skin of the patient.

15. The method of claim 13, wherein the detector is a resonator.

16. The method of claim 13, further comprising mechanically relaying the sound detected by the detector to the speaker.

17. The method of claim 13, further comprising electronically relaying the sound detected by the detector to the speaker.

18. The method of claim 17, wherein the detector comprises a microphone.

19. The method of claim 13, wherein the speaker continuously amplifies the sound detected by the detector.

20. The method of claim 19, further comprising producing an alarm with the speaker when no sound (or sound below a threshold volume) is detected by the detector.

21. The method of claim 13, wherein attaching the detector to the skin of the patient comprises attaching the detector to an extremity of the patient.

22. A method of monitoring a pulse, the method comprising:
sending an ultrasonic wave;
detecting a reflection of the ultrasonic wave;
converting the reflection of the ultrasonic wave into an electrical signal; and
relaying the electrical signal to the speaker to signify a pulse.
